(19) United States
(12) Patent Application Publication
(10) Pub. No.: US 2007/0072174 A1
(43) Pub. Date: Mar. 29, 2007

(54) Bioreporter for detection of microbes

(76) Inventors: Gary S. Sayler, Blaine, TN (US); Steven A. Ripp, Knoxville, TN (US); Alice Layton, Knoxville, TN (US)

Correspondence Address: RUDEN, MCCLOSKY, SMITH, SCHUSTER & RUSSELL, P.A., 222 LAKEVIEW AVE, SUITE 800, WEST PALM BEACH, FL, 33401-6112, US

(21) Appl. No.: 11/237,517

(22) Filed: Sep. 28, 2005

(51) Int. Cl. C12Q 1/70; C12Q 1/68

(52) U.S. Cl. 435/5; 435/6; 977/802

(57) ABSTRACT

A recombinant phage system has been developed for the rapid detection of bacteria, particularly fecal coliform indicator bacteria. The systems of the invention link phage infection events to quorum sensing signal molecule biosynthesis and bioluminescent bioreporter induction, facilitating the detection of pathogens that may be present in low numbers. The phage-based systems of the invention maintain specificity for the pathogen while still producing significant signal amplification for sensitive and quantitative detection. The systems require only the combination of sample with phage and bioreporter organisms; no extraneous addition of any substrates or user intervention of any kind is necessary, making this approach significantly less technical than standard molecular or immunological methods.

### STATEMENT AS TO FEDERALLY SPONSORED RESEARCH

The invention was made with U.S. government support under grant number NAG9-1424 awarded by the NASA Advanced Environmental Monitoring and Control Program and under grant number 2001-02996 awarded by the United States Department of Agriculture. The U.S. government may have certain rights in the invention.

### FIELD OF THE INVENTION

The invention relates generally to the fields of microbiology, environmental testing, and food safety. More particularly, the invention relates to systems, compositions and methods for measuring bacterial contamination in a sample.

### BACKGROUND

In 1987, Ulitzur and Kuhn (“Introduction of lux genes into bacteria, a new approach for specific determination of bacteria and their antibiotic susceptibility. In: Scholmerich J, Andreesen R. Kapp A, Ernst M. Woods (WG (eds) Bioluminescence and Chemiluminescence: New Perspectives. John Wiley & Sons, New York, 1987, p. 463-472) reported a novel pathogen detection method that coupled the specificity of bacteriophages (phages) for their unique bacterial hosts with bioluminescent signalling. They cloned the luxAB encoded luciferase genes from Vibrio fischeri into the phage lambda genome. Upon infection, the luxAB genes were transduced into Escherichia coli, thus endowing these host cells with a bioluminescent phenotype visible upon addition of a requisite aldehyde substrate. This technique has since been applied to other phage for specific, low-level (10-1000 cells) detection of Listeria monocytogenes (Loessner et al. Appl Environ Microbiol 62, 1133-1140, 1996), Salmonella typhimurium (Chen et al., J Food Protect 59, 908-914, 1996), E. coli O157:H7 (Waddell et al., FEMS Microbiol Lett 182, 285-289, 2000), enteric bacteria (Kodikara et al., FEMS Microbiol Lett 83, 261-266, 1991), and Staphylococcus aureus (Pagotto et al., Bacterial Quality Raw Milk, 9601, 152-156, 1996) within a variety of food matrices. The firefly luciferase (luc) (Sarkis et al., Mol Microbiol 15, 1055-1067, 1995), ice nucleation (inaW) (Wolber et al., Trends in Biotechnology 8, 276-279, 1990), beta-galactosidase (lacZ) (Goodridge et al., Food Res Int 35, 863-870, 2002), and green fluorescent protein (gfp) (Funatsu et al., Microbiol Immunol 46, 365-369, 2002; and Oda et al., Appl Environ Microbiol 70, 527-534, 2004) genes have similarly been incorporated into bacteriophages for the detection of foodborne pathogens such as Mycobacterium, Salmonella, and E. coli. Reporter phages have also been labeled with a variety of fluorescent dyes for bacterial-specific tagging (Mosier-Boss et al., Appl Spectrosc 57, 1138-1144, 2003) and combined with immunomagnetic separation for rapid capture, concentration, and identification of bacterial targets (Goodridge et al., Appl Environ Microbiol 65, 1397-1404, 1999; and Favrin et al., Appl Environ Microbiol 67, 217-224, 2001). In addition, bacteriophage in their unadorned native form have been used for decades in phage typing schemes to identify foodborne as well as clinical bacterial isolates (Stone, Science 298: 728-731, 2002). Although exploitation of phage specificity for bacterial monitoring has potential for foodborne pathogen monitoring, current phage assay systems require the addition of substrate or specialized monitoring equipment that is not adaptable to the real-time, on line monitoring format desired by the food industry.

The key technological metrics required by the food industry for effective detection and monitoring of bacterial pathogens are sensitivity, specificity, speed, simplicity, and cost-effectiveness. Portability can also be added to this list as quality control testing begins to move from the centralized laboratory to strategic on-the-spot monitoring within the production line itself. The traditional methods of selective sample enrichment followed by any number of morphological, biochemical, or serological tests offer little in the way of rapidity, often requiring several days from initial sampling to final analysis. The introduction of nucleic acid-based detection technologies affords some significant increases in response times as well as improved sensitivity and specificity, but the complexity and costs involved in routine analysis limits their universal application.

### SUMMARY

A recombinant bacteriophage-based system has been developed for the rapid detection of a particular species of bacteria, particularly fecal coliform indicator bacteria, in a sample. The system described herein involves the luxCDABE operon, its regulatory genes luxI and luxR, and a phage chosen based on its specificity for the desired target bacterium that is engineered to contain the luxI gene within its chromosome. The luxI-encoded LuxI protein is responsible for generation of a specific acyl-homoserine lactone (AHL) signaling molecule referred to as an autoinducer within the target bacterium. Upon infection of the target bacterium by the recombinant bacteriophage, luxI is inserted into the target bacterium and expressed, thereby creating a cell that actively synthesizes autoinducer. As the autoinducer molecules diffuse into the extracellular environment, they are detected by an AHL-specific bioluminescent bioreporter that contains the luxR and luxCDABE genes. The luxAB component of this operon encodes a bacterial luciferase that generates bioluminescence when provided with oxygen, FMNH₂, and an aldehyde substrate synthesized by the luxCDE gene complex. The interaction of autoinducer with LuxR protein stimulates luxCDABE expression and the bioreporter generates a light signal at 490 nm. As the concentration of AHL autoinducer increases, so does the number of LuxR binding episodes, and an autoamplified quorum sensing loop is established that results in the generation of bioluminescence in a cell density-dependent manner. Thus, the initial phage infection event yields an autoamplified chemical signature that is sensed and communicated through bioluminescent bioreporter signal induction.

The phage-based assays described herein overcome a number of limitations inherent to conventional bioreporter systems. Conventional reporters require the addition of an inducing substrate or other external manipulation to initiate signaling. The embodiments of the invention described herein do not require the addition of substrate or other reagents, only the addition of sample. Another advantage provided by the phage detection systems described herein involves the maximal amplification of the phage infection event using quorum sensing autoinducer signaling. Additionally, the luxI gene is only 258 bp in size, as compared to other previously used phage reporter genes such as luxAB, lacZ, or luc that range from 1600-3000 bp. This allows several luxI genes to be inserted into the phage genome such that each phage infection event can result in multiple luxI transcriptions, rather than the single phage/single reporter transcription events exhibited by other phage reporters, resulting in greater signal amplification per target cell.

Yet another advantage is that the host cell itself is not responsible for generating the final signal. In real-world samples, target (i.e., host) cells are typically not in an optimal growth state, and expecting such cells to divert their limited resources to metabolically intense pathways such as bioluminescence production is not feasible or favorable. In the embodiments described herein, the host cell only needs to transcribe luxI; the sensing of the resultant autoinducer signal is accomplished by ancillary healthy bioreporters. Further, since the bioreporter is a secondary component of the assay, it can be added in any quantity desired (within reason since there will be competitive growth between the bioreporters and target cells). Thus, the number of bioreporters is not limited to the number of target cells, as is the case when using the host cell as the bioreporter cell. Having many bioreporters better ensures signal detection and permits accumulative responses.

Accordingly, the invention features a method for detecting a target bacterium in a sample. This method includes the steps of: (a) contacting the sample with a recombinant bacteriophage that is capable of infecting the target bacterium, the recombinant bacteriophage including a nucleotide sequence encoding a molecule capable of upregulating synthesis of at least one autoinducer molecule in the target bacterium; (b) contacting at least a portion of the sample that has been contacted with the recombinant bacteriophage with at least one bioreporter bacterium including (i) a receptor capable of specifically binding the at least one autoinducer molecule and (ii) a nucleic acid encoding a reporter molecule; (c) placing the at least a portion of the sample that has been contacted with the at least one bioreporter bacterium under conditions that promote (i) the expression of and diffusion of the at least one autoinducer molecule from the target bacterium and (ii) the uptake of the at least one autoinducer molecule by the at least one bioreporter bacterium; and (d) detecting expression of the reporter molecule in the at least one bioreporter bacterium, wherein expression of the reporter molecule indicates that the target bacterium was present in the sample. In this method, the reporter molecule can include LuxA and LuxB and binding of the at least one autoinducer molecule to the receptor can upregulate expression of the nucleic acid encoding a reporter molecule. The molecule capable of upregulating synthesis of at least one autoinducer molecule in the target bacterium can be LuxI, and the receptor that specifically binds the at least one autoinducer can be LuxR.

In methods of the invention, the at least one bioreporter bacterium can further include a nucleic acid encoding LuxC operably linked to at least one promoter, a nucleic acid encoding LuxD operably linked to at least one promoter, and a nucleic acid encoding LuxE operably linked to at least one promoter. The amount of reporter molecule expression can be proportional to the amount of target bacteria in the sample. The target bacterium can be a food pathogen (e.g., Escherichia coli). The sample can be water, food, and water that has contacted food. The recombinant bacteriophage can be phage lambda and the at least one bioreporter bacterium can be Escherichia coli. The autoinducer molecule can be an acyl-homoserine lactone (e.g., N-3-(oxohexanoyl)-L-homoserine lactone). A recombinant bacteriophage can further include at least three copies (e.g., 2, 3, 4, 5, 6, 7, 8, 9, 10) of the nucleotide sequence encoding a molecule capable of upregulating synthesis of at least one autoinducer molecule.

In another aspect, the invention features a kit for detecting a target bacterium in a sample. This kit includes (a) a recombinant bacteriophage that is capable of infecting the target bacterium, the recombinant bacteriophage including a nucleotide sequence encoding a molecule capable of upregulating synthesis of at least one autoinducer molecule in the target bacterium; and (b) instructions for using the recombinant bacteriophage in conjunction with at least one bioreporter bacterium including (i) a receptor capable of specifically binding the at least one autoinducer molecule and (ii) a nucleic acid encoding a reporter molecule. This kit can further include (c) at least one bioreporter bacterium including (i) a receptor capable of specifically binding the at least one autoinducer molecule and (ii) a nucleic acid encoding a reporter molecule, wherein expression of the reporter molecule indicates the presence of the target bacterium in the sample. The reporter molecule can include LuxA and LuxB, the molecule capable of upregulating synthesis of at least one autoinducer molecule in the target bacterium can be LuxI, and the receptor that specifically binds the at least one autoinducer molecule can be LuxR. The target bacterium can be a food pathogen (e.g., Escherichia coli). The recombinant bacteriophage can be phage lambda, and the at least one bioreporter bacterium can be Escherichia coli. The at least one bioreporter bacterium can be resistant to infection by the recombinant bacteriophage. The autoinducer molecule can be an acyl-homoserine lactone (e.g., N-3-(oxohexanoyl)-L-homoserine lactone). The molecule that is capable of upregulating synthesis of at least one autoinducer molecule in the target bacterium can upregulate synthesis of a plurality of autoinducer molecules in the target bacterium. Binding of the at least one autoinducer molecule to the receptor can upregulate expression of the nucleic acid encoding the reporter molecule.

Another kit within the invention is a kit for detecting a target bacterium in a sample. This kit includes a solid substrate having a plurality of bioreporter bacteria disposed thereon, each bioreporter bacterium including (i) a receptor capable of specifically binding the at least one autoinducer molecule and (ii) a nucleic acid encoding a reporter molecule, the bioreporter bacteria being in operable proximity to an integrated circuit for detecting and quantitating expression of the reporter molecule, and instructions for use of the kit with a recombinant bacteriophage that is capable of infecting the target bacterium, the recombinant bacteriophage including a nucleotide sequence encoding a molecule capable of upregulating synthesis of at least one autoinducer molecule in the target bacterium. The molecule capable of upregulating synthesis of at least one autoinducer molecule in the target bacterium can upregulate synthesis of a plurality of autoinducer molecules in the target bacterium. The solid substrate can be a microchip and the kit can be portable. The amount of reporter molecule expression can be proportional to the amount of target bacteria in the sample.

Yet another kit within the invention is a kit for detecting a target bacterium in a sample. This kit includes a solid substrate having a plurality of bioreporter bacteria disposed thereon, each bioreporter bacterium including (i) a receptor capable of specifically binding the at least one autoinducer molecule and (ii) a nucleic acid encoding a reporter molecule, the bioreporter bacteria in operable proximity to at least one photodetector for detecting expression of the reporter molecule, the photodetector in operable engagement with at least one processor for storing information pertaining to the expression of the reporter molecule.

Unless otherwise defined, all technical terms used herein have the same meaning as commonly understood by one or ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote, polymerase chain reaction (PCR) products, nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

As used herein, “protein” or “polypeptide” are used synonymously to mean any peptide-linked chain of amino acids.

By the terms “LuxR protein,” LuxR polypeptide,” or simply “LuxR” is meant an expression product of a luxR gene; or a protein that shares at least 65% (but preferably 75, 80, 85, 90, 95, 96, 97, 98, or 99%) amino acid sequence identity with the sequence having accession number M19039 and displays a functional activity of LuxR. Similarly, by the terms “LuxI protein,” LuxI polypeptide,” or simply “LuxI” is meant an expression product of a luxI gene; or a protein that shares at least 65% (but preferably 75, 80, 85, 90, 95, 96, 97, 98, or 99%) amino acid sequence identity with the sequence having accession number M19039 and displays a functional activity of LuxI.

By the terms “bioreporter” and “bioreporter bacterium” is meant a bacterial cell having a nucleic acid encoding at least one Lux protein (e.g., LuxR, LuxA, LuxB, LuxC, LuxD, LuxE) and that is resistant to infection by a recombinant bacteriophage.

As used herein, the terms “target bacterium” and “host bacterium” mean a bacterial cell that is to be detected in a sample using a system of the invention.

25. As used herein, the term “vector” refers to a nucleic acid molecule capable of transporting another nucleic acid to which it has been linked. Vectors capable of directing the expression of genes to which they are operatively linked are referred to herein as “expression vectors.”

A first nucleic acid sequence is “operably” linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame. Operably linked nucleic acids can also be non-contiguous.

A “homolog” of a Vibrio fischeri luxR gene is a gene sequence encoding a LuxR polypeptide isolated from a bacterium other than V. fischeri. Similarly, a “homolog” of a native LuxR polypeptide is an expression product of a luxR homolog. A “homolog” of a V. fischeri luxI gene is a gene sequence encoding a LuxI polypeptide isolated from a bacterium other than V. fischeri. Similarly, a “homolog” of a native LuxI polypeptide is an expression product of a luxI homolog.

As used herein, a “reporter molecule” is any molecule whose expression in a cell can be modulated in response to an autoinducer molecule. A reporter molecule can be, for example, a multi-component complex, or a component of a multi-component complex. Examples of reporter molecules include bacterial luciferase, green fluorescent protein, and firefly luciferase, as well as colorimetric, chemiluminescent, and electrochemical signals.

Although systems, methods and materials similar or equivalent to those described herein can be used in the practice or testing of the present invention, suitable systems, methods and materials are described below. All publications, patent applications, patents, and other references mentioned herein are incorporated by reference in their entirety. In the case of conflict, the present specification, including definitions, will control. In addition, the particular embodiments discussed below are illustrative only and not intended to be limiting.

### BRIEF DESCRIPTION OF THE DRAWINGS

FIG. 1 is a schematic illustration of a reporter phage assay for targeted detection of bacterial pathogens.

FIG. 2 is a diagram illustrating the genetic construction of the λ_{luxI} reporter bacteriophage.

FIG. 3 is a diagram illustrating the genetic construction of N-3-(oxohexanoyl)-L-homoserine lactone (OHHL)-specific bioluminescent bioreporter E. coli OHHLux.

FIG. 4 is a schematic illustration of components of a nanostructured bioluminescent bioreporter integrated circuit biosensor.

### DETAILED DESCRIPTION

The invention provides a system for detecting a target bacterium in a sample. In the experiments described herein, the luxI gene from V. fischeri was inserted into the lambda phage genome to construct a phage-based biosensor assay for the general detection of E. coli. In a quorum sensing event, autoinducer signaling molecules synthesized upon phage infection of the E. coli target bacterium are detected by an autoinducer-specific bioluminescent bioreporter based on the luxCDABE gene cassette. The assay generates target-specific visible light signals with no requisite addition of extraneous substrate. Rather than sensing a single biological entity, an amplified chemical signature manifested from that entity is detected, thereby permitting detection of very low density target populations. When used in conjunction with a microelectronic luminometer chip, the bioluminescent signaling event resulting from reporter phage infection can be measured within a miniaturized, portable, self-contained format.

The below described preferred embodiments illustrate adaptations of these systems, compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 2003 (with periodic updates). Various techniques using PCR are also described in methodology treatises, e.g., PCR Protocols, 2^{nd} edition, Bartlett, John, M. S. (ed.) and Stirling, David (ed.), Humana Press: Totowa, N.J., 2003; and PCR Primer, 2^{nd} edition, Dieffenbach, Carlos (ed.) and Dveksler, Gabriela S. (ed.), Cold Spring Harbor Laboratory Press, Cold Spring Harbor, N.Y., 2003. PCR-primer pairs can be derived from known sequences by known techniques such as using computer programs intended for that purpose (e.g., Primer, Version 0.5, ©1991, Whitehead Institute for Biomedical Research, Cambridge, Mass.). Methods for performing real-time PCR are also known in the art and are described in, for example, Belanger et al., J. of Clinical Microbiology 41:730-734, 2003; Winter et al., Curr Pharm Biotechnol. 2:191-197, 2004; and Mackay, I. M., Clin Microbiol Infect. 10:190-212, 2004. lux-based bioluminescent bioreporters are described, for example, in Daunert et al., Chem Rev. 100:2705-2738, 2000; and Keane et al., J Microbiol Methods 49:103-119, 2002.

### Quorum Sensing Cell-to-Cell Communication Networks

The detection systems described herein involve a quorum sensing mechanism that allows a bioreporter cell to emit a detectable signal based on the infection of a host cell by a recombinant phage of the invention. Quorum sensing refers to a cell-to-cell communication network based on the synthesis of diffusible autoinducer molecules. See, for example, Miller et al., Annu Rev Microbiol 55, 165-199, 2001. Autoinducers increase in concentration as a function of cell density, and, upon reaching a minimum threshold value, concertedly induce a particular gene or set of genes throughout the bacterial population, allowing the entire population to respond in unison to changing environmental conditions.

### System for Detecting Target Bacteria in a Sample

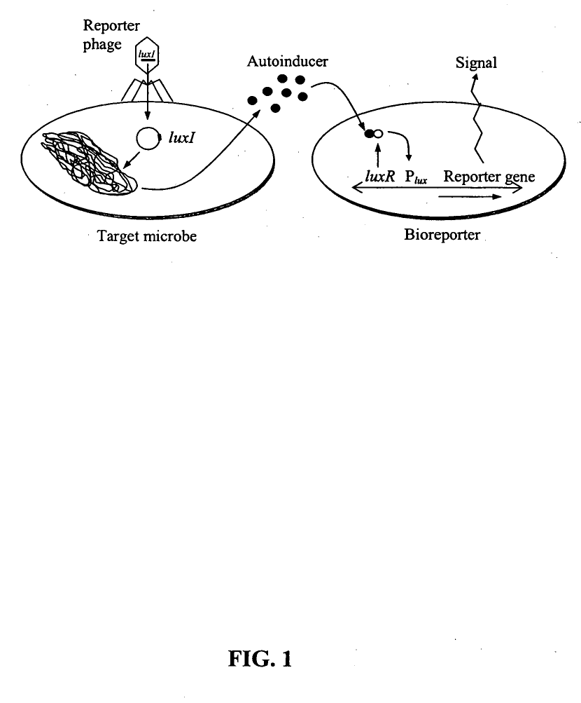

FIG. 1 shows a recombinant phage-based reporter assay for detecting bacteria (i.e., target bacteria) in a sample. A sample to be tested using the system of FIG. 1 can be water, food (e.g., meat, vegetables, fruit, processed food, poultry, eggs, milk, or cheese), or water that has contacted food. The target bacterium can be a food pathogen such as a fecal coliform indicator bacteria. Examples of fecal coliform indicator bacteria include E. coli, Bacteroides, fecal coliforms, and fecal enterococci (Tendolkar et al., Cell Mol Life Sci. 60:2622-2636, 2003; Simpson et al., Environ Sci Technol. 36:5279-5288, 2002). In the experiments described below, E. coli XL1-Blue was used as a model bacterial pathogen. However, the assay can be used to detect any target bacterium that can be infected by a bacteriophage. A non-exhaustive list of additional target bacteria includes enteric bacteria, Listeria monocytogenes, Salmonella typhimurium, Staphylococcus aureus, Yersinia, and Mycobacterium.

The choice of bacteriophage to be used in the system is dependent upon the target bacterium to be detected. For example, if the target bacterium is Salmonella, the bacteriophage is one capable of infecting Salmonella (e.g., bacteriophage P22). In the experiments described below, phage lambda was chosen for the detection of E. coli. Examples of additional phage include bacteriophage L, bacteriophage K139, bacteriophage Ms6, bacteriophage MS2, bacteriophage P4, bacteriophage T4, bacteriophage phi29, bacteriophage phi22, bacteriophage phiC31, bacteriophage M13, bacteriophage phiX174, and mycophage. A non-exhaustive list of phage-host combinations is presented below in Table 1. Once a bacteriophage is chosen, it is modified to contain a nucleotide sequence encoding an enzyme that induces synthesis of at least one autoinducer molecule. In the system described herein, modified bacteriophage contain a sequence encoding LuxI protein, an enzyme that induces synthesis of autoinducer molecules. A preferred luxI gene for use in the embodiments described herein is from V. fischeri because this gene has been well characterized genetically. However, any suitable luxI homolog can also be used (see Table 1 of Miller, M. B., and B. L. Bassler, Annu. Rev. Microbiol. 55:165-199, 2001). Other nucleic acids that encode enzymes that induce synthesis of autoinducer molecules are known in the art (see, e.g., Bertani, I., and V. Vittorio, Applied and Environmental Microbiology, 70:5493-5502, 2004; Surette et al., Proc. Natl. Acad. Sci. USA, 96:1639-1644, 1999; Zhu et al., Journal of Bacteriology, 180:5398-5405, 1998; Fuqua, W. C., and S. C. Winans, Journal of Bacteriology, 176:2796-2806, 1994; and Shiner et al., Biol. Proced. Online 6(1):268-276, 2004) and may be used in the invention.

Upon infection of the phage's specific host (i.e., the target or host bacterium that is to be detected), the nucleotide sequence encoding LuxI is inserted into the host chromosome where it is transcribed, thereby synthesizing LuxI. The LuxI protein aids in the production of autoinducer which diffuses from the target bacterium into a neighboring bioreporter cell having nucleic acids encoding LuxR, LuxA, LuxB, LuxC, LuxD, and LuxE. Once inside the bioreporter cell, the AHL (e.g., OHHL) molecules interact with (i.e., bind to) the LuxR regulatory protein, a receptor protein that when bound to an inducer molecule, dimerizes and binds to a response element (e.g., Lux box) located in the promoter region of target genes, activating expression of these genes. Binding of the AHL molecules to LuxR triggers luxCDABE transcription in the bioluminescent bioreporter cell to generate bioluminescence, indicating the presence of the target bacterium in the sample. Many LuxR homologs exist and may be used in the invention. Analagous receptor proteins from other systems (e.g., QscR, LasR, and RhlR from P. aeruginosa, TraR from Agrobacterium, etc.) are known as well. See, e.g., Slock et al., Journal of Bacteriology, 172:3974-3979, 1990; and Stevens A. M., and E. P. Greenberg, Transcriptional Activation by LuxR. In: Cell-Cell signaling in bacteria. Edited by Dunny G M, Winans S C. Washington, D.C.: American Society for Microbiology, 1999:231-242.

In the experiments described below, bioreporters that specifically sense the autoinducer OHHL were used. However, any suitable autoinducer can be used in the system. A number of AHL autoinducers are known including N-3-(oxohexanoyl)-L-homoserine lactone, N-butyryl-HSL, and N-(3-oxododecanoyl)-HSL (Surette et al., Proc. Natl. Acad. Sci. USA 96:1639-1644, 1999; M. R. Parsek and E. P. Greenberg, Proc. Natl. Acad. Sci. USA 97:8789-8793, 2000). See Table 2 for a non-exhaustive list of autoinducer molecules.

TABLE 1

| Host Strain | Bacteriophage |
|---|---|
| Staphylococcus aureus NCIMB 8588 | phage NCIMB 9563 |
| S. aureus V8 (ATCC 49775) | φPVL (temperate) |
| S. aureus | 187, Twort, Phage library, φETA, φSLT |
| Salmonella typhimurium DB7156 and LT2 | Felix O-1, IRA, MB78, P22, SP6, 9NA, |
| | Sapphire |
| Salmonella enteritidis | φSJ2 |
| Pseudomonas aeruginosa NCIMB 10548 | phage NCIMB 10116 and 10884 (E79), D3, |
| | φCTX |
| Listeria monocytogenes | A511, A118, phage library |
| Escherichia coli O157:H7 | φV10, LG1 (17), KH1, KH4, KH5 |
| E. coli K12 derivatives (JM101, AB1157, | HK97, HK022, N15, λimm21 |
| JC11801) | |
| E. coli JM105 | M13 |
| Streptococcus thermophilus | DT1, φO1205 |
| Mycobacterium tuberculosis , smegmatis, leprae | D29, phAE85 |
| Mycobacterium smegmatis, bovis | L5, Bxb1 |
| Bacillus subtilis | SPP1, SPβc2, B103, PVA, Φ105 |
| Bacillus cereus | 12826 (ATCC12826-B1), Bastille, TP21 |
| Bacillus thuringiensis | KK-88, J7W-1 |
| Campylobacter jejuni, coli | Phage library |
| Streptomyces | φC31 |
| Shigella flexneri | SfX, SfV, SfII, Sf6 |
| Yersinsia enterocolitica serotype O: 3 | φYeO3-12, PY54 |
| Yersinia pestis | Phage II, L-413-C, 1701 |
| Clostridium botulinum | c-st, d-16o, c-st, d-1837, d-sa, d-1, CE§, C |
| | and D, alpha-2 |
| Clostridium perfringens | phi29 and phi59, p1-p24, S9, PF1-4, c1, c3-5, |
| | φ3626 |
| Brucella melitensis | ATCC 23456-B1 (BK-2) |
| Brucella abortus | ATCC 23448-B1 (TBLISI), ATCC 17358-B1 |
| | (212/XV) |
| Brucella suis | Weybridge, M51, S708, Th, Unlisted (Gargani, G. |
| | 1965, Action of bacteriophages on Brucella |
| | abortus and on Brucella suis. Boll Ist Sieroter |
| | Milan 44: 189-201) |
| Brucella canis | R/C |
| Vibrio cholera | ATCC 14100 B1-4 (138, 145, 149, 163), ATCC |
| | 51352 B1-10 (N-4, S-5, S-20, M-4, D-10, I, II, |
| | III, IV, V) |
| Pseudomonas pseudomallei | Unlisted (Denisov, I. and V. Kapliev, |
| | Mikrobiol Z. 57: 53-56, 1995), PP19, PP23, |
| | PP33, Unlisted (Grishkina and Merinova, |
| | Mikrobiol Z. 55: 43-47, 1993), Unlisted |
| | (Denisov and Kapliev, Mikrobiol. Zh. 53: 66-70, |
| | 1991) |
| Francisella tularensis | PRDI |
| Bacillus anthracis | Φ20, Ap50, CN 18-74 and CN 35-18, Unlisted |
| | (Nagy, E., Acta Microbiologica Academiae |
| | Scientiarum Hungaricae 21: 257-263, 1974), |
| | Tg13ant, CP54, 27cr, 27tl, 29cg, phage W, |
| | cherry |
| Burkholderia mallei | E125 |

A preferred bioreporter bacterium for use in the system is a bacterium that contains a chromosomal insert of the luxR regulatory gene and the complete luxCDABE gene cassette from V. fischeri and that is specific for the autoinducer that is produced by the target bacterium. Any suitable bacteria can be used as the bioreporter cells but the bacteria used should be resistant to (i.e., cannot be infected by) the recombinant phage that is being used to infect the target (i.e., host) cells. In the experiments described herein, E. coli OHHLux was used as the bioreporter bacterium. E. coli OHHLux is specific for OHHL and is resistant to infection by phage lambda. Although the experiments described herein involve the use of luxCDABE from V. fischeri, the lux cassette can be from other luminescence-producing bacteria including Photorhabdus luminescens or Vibrio harveyi. In addition, insect luciferase (luc from the firefly or click-beetle) can be used. Besides luminescence, AHL-specific bioreporters can also be made to generate signals that are fluorescent (using green fluorescent protein) or derivatives that fluoresce in cyan, red, or yellow wavelengths as well as aequorin or uroporphyrinogen III methyltransferase (UMT)). Colorimetric (lacZ, xylE, bla), chemiluminescent, and electrochemical signals can also be implemented within the invention.

In a preferred system of the invention, the target bacterium that is infected by the recombinant bacteriophage produces a plurality of diffusible autoinducer molecules which permeate the extracellular environment. The plurality of autoinducer molecules cross the cell membranes of a plurality of bioreporter cells inducing bioluminescence in the plurality of bioreporter cells, resulting in a cascade effect that ultimately generates intense bioluminescent light. Generating such levels of bioluminescence aids in the measuring of the bioluminescence and therefore in the detection and quantification of the target bacteria.

### Vectors/Regulatory Elements

Natural or synthetic nucleic acids encoding LuxI, LuxR, LuxA, LuxB, LuxC, LuxD and LuxE can be incorporated into vectors and/or operably linked to one or more regulatory elements for delivery into bacteriophage or bacteria. Examples of regulatory elements include promoters, initiation sites, response elements, and termination signals. For example, a nucleic acid encoding LuxI operably linked to a promoter is inserted into the bacteriophage genome via ligating the nucleic acid into an appropriate cloning vector (e.g., Lambda ZAP II cloning vectors by Stratagene, LaJolla, Calif.) and packaging into phage heads using a suitable packaging reagent (e.g., Gigapack III Gold packaging extract by Stratagene). Nucleic acids encoding LuxR, LuxA, LuxB, LuxC, LuxD and LuxE operably linked to a promoter can be integrated into the genomes of the bacteria of the invention, or they may exist episomally in the bacteria. Any suitable vector that includes a replication system and sequences that are capable of transcription and translation of a polypeptide-encoding sequence in a given host cell may be used. Examples of expression and/or cloning vectors include pCR2.1-TOPO (Invitrogen, Carlsbad, Calif.), pCR4-TOPO (Invitrogen), pLEX, pYES2.1, pCR-XL-TOPO, pGEM, EZ::TN pMOD, and Lambda ZAP II (Stratagene), as well as variations thereof. Expression vectors preferably include regulatory elements that facilitate expression of a polypeptide in a host cell. For the practice of the present invention, conventional compositions and methods for preparing and using vectors and host cells are employed, as dicussed, e.g., in Sambrook et al., supra or Ausubel et al., supra.

To achieve appropriate levels of LuxI, LuxR, LuxA, LuxB, LuxC, LuxD, and LuxE proteins, any of a number of promoters suitable for use in the selected host cell may be employed. For example, constitutive promoters of different strengths can be used to express the LuxI, LuxR, LuxA, LuxB, LuxC, LuxD, and LuxE proteins. Inducible promoters can also be used to express the LuxI, LuxR, LuxA, LuxB, LuxC, LuxD, and LuxE proteins. To achieve regulated expression of LuxA, LuxB, LuxC, LuxD, LuxE and proteins in bioreporter cells and expression of LuxI in the target bacteria, the left arm promoter (P_{L}) of phage lambda is preferred because it is genetically well characterized. However, any promoter known to function in bacterial cells may be used

### Detecting a Target Bacterium in a Sample

The invention encompasses methods for detecting the presence of a target bacterium in a sample as well as methods for quantifying the amount of target bacteria in a sample. Such methods are useful for evaluating bacterial contamination of water, food, and air. An exemplary method for detecting a target bacterium in a sample involves providing a sample; providing a recombinant bacteriophage having a nucleotide sequence encoding LuxI and capable of infecting the target bacterium, the LuxI inducing the production of at least one autoinducer molecule in the target bacterium when the bacteriophage infects the target bacterium; infecting the target bacterium with the recombinant bacteriophage, resulting in expression of and diffusion of the at least one autoinducer molecule; and allowing the at least one autoinducer molecule to cross the cell membrane of at least one bioreporter bacterium including nucleic acids encoding LuxR, LuxA, LuxB, LuxC, LuxD, and LuxE. In this method, binding of the at least one autoinducer molecule to at least one LuxR activates expression of the nucleic acids encoding LuxA, LuxB, LuxC, LuxD, and LuxE, resulting in bioluminescence. The generated bioluminescence indicates the presence of the target bacterium.

In a typical method, LuxI induces the production of a plurality of autoinducer molecules when the recombinant bacteriophage infects the target bacterium. In this method, the plurality of autoinducer molecules cross the cell membranes of a plurality of bioreporter bacteria inducing bioluminescence in the plurality of bioreporter cells. If the sample contains no target bacteria, then no autoinducer molecules are produced and no bioluminescence is generated.

In an exemplary method of quantifying the level of target bacteria in a cell, the steps of detecting a target bacterium in a sample are first performed. These steps are followed by the steps of measuring the level of bioluminescence, and correlating the level of bioluminescence with the quantity of target bacteria in the sample. In the exemplary embodiments described herein, the level of bioluminescence is proportional to the quantity of target bacteria in the sample. Devices for measuring bioluminescence levels are described below.

### Bioluminescent Bioreporter Integrated Circuits (BBICs)

The phage-based detecting systems described herein can be used in combination with a means for measuring luminescence emitted by the bioreporter cell when in the presence of an analyte (e.g., autoinducer molecule) or pathogen (e.g., E. coli). Typically, an autoinducer molecule (e.g., OHHL) diffuses across the cell membranes of the bioreporter cells and activates transcription of the luxCDABE gene cassette (or other nucleic acid(s) encoding a reporter molecule) in the bioreporter cells, initiating a bioluminescent response that can be quantified by an electronic, optical, or mechanical inducer. In some applications, the bioreporter cells may be incorporated in a BBIC, a whole-cell integrated chemical sensor. Generally, the cells are maintained in close proximity to the integrated circuit (IC) of the BBIC. The IC portion of the BBIC detects and quantifies the luminescence and reports this data to (in some cases wirelessly) a central data collection location. The major components of the IC are the integrated photodetectors, the signal processing, and the wireless circuitry. These major components are described in, for example, Simpson et al., Trends in Biotechnology, 16:332-338, 1998; and Bolton et al., Sens. Actuators B, 85:179-185, 2002. Information comes into the system when the autoinducer increases or upregulates expression of the luxCDABE cassette in the bioreporter cells. The system measures and reports the magnitude of the upregulation. Compatible electronic ICs and biosensor devices are described in U.S. Pat. Nos. 6,255,708 and 6,544,729. CMOS microluminometers that may be used in the invention are described in, for example, Simpson et al., Sens. Actuators B, 72:134-140, 2001; and Bolton et al., Sens. Actuators B., 85:179-185, 2002.

In some applications, the bioreporter cells are used in portable bioluminescence detectors. Such detectors that may be used outside the laboratory are made using IC optical transducers that directly interface with cells (e.g., BBICs, Bolton et al., Sens. Actuators B, 85:179-185, 2002; and Nivens et al., J. Appl. Microbiol. 96:33-46, 2004). These BBICs are contained within an approximate 1 cm³ area and include two main components: photodetectors for capturing the on-chip bioluminescent bioreporter signals and processors for managing and storing information derived from bioluminescence. The photodetector can be inlaid as sectional modules to allow for independent sensing of bioluminescence from multiply adhered bioreporters. Radio frequency (RF) transmitters can also been incorporated for wireless data relay. Since the bioreporter and biosensing elements are completely self-contained within the BBIC, operational capabilities are realized by simply exposing the BBIC to the desired test sample (Ripp et al., J Ind Microbiol Biotechnol. 30:636-642, Epub 2003) and the corresponding recombinant bacteriophage.

In applications utilizing bioreporter technology as a methodology for wide area target contaminant monitoring, a single microchip OASIC transducer that couples directly to bioreporter matrices is useful. This transducer provides a complete, standalone detection system for wide area monitoring of chemical and biological agents. A test bed of integrated circuits for replicate measurement of induced bioreporter bioluminescence has been developed. The test bed contains various static and flow-through modules that suspend the bioreporters directly above the integrated circuit luminometer. In another embodiment, the functional biochip can include a standalone disposable unit containing everything necessary for independent sensing of pathogenic agents.

Using the integrated circuit test bed, phage-amplified bioluminescent bioreporters can be tested within light-tight enclosures containing glass vials or flow-through chambers. The OHHLux bioluminescent bioreporters, luxI-integrated phage, and target pathogens in buffered media or tap water are added in optimized ratios as determined from the experiments described below. Samples are then exposed to the BBIC in the flow-cell format for continuous, real-time monitoring of bioluminescent signals (following methods reported by Nivens et al., J. Appl. Microbiol. 96:33-46, 2004). Individual samples are also removed periodically for single-point bioluminescent measurements in the glass vials. This system is useful for direct microbiological analysis of water samples, for example.

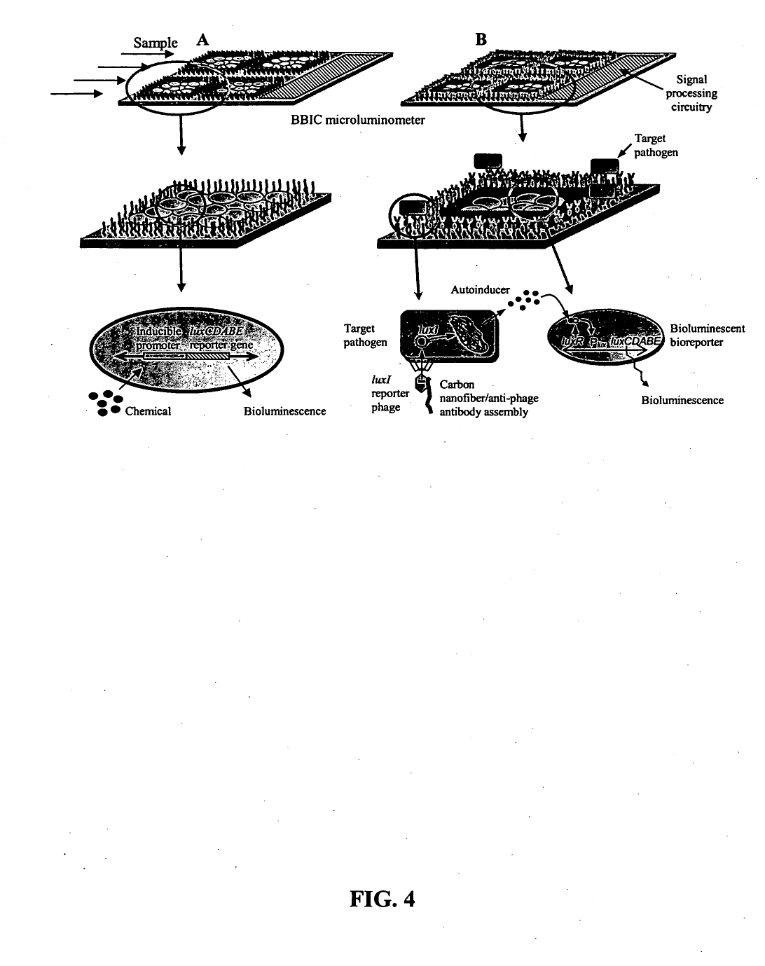

Yet another embodiment of the phage- and BBIC-based system is a multiplexed sensor capable of simultaneously monitoring multiple pathogens. To achieve this, the BBIC is designed to hold several phage reporter systems, each unique to a targeted pathogen. Nanofiber arrays are synthesized on a chip to create caged structures capable of containing each bioreporter population (FIG. 4). In FIG. 4A, the biosensor for chemical contaminant detection includes nanostructurally caged bioluminescent bioreporters segmented on a microluminometer chip. The genetic design of each bioreporter population allows for sensing and response to a unique target chemical or chemical class, thereby permitting multiplexed detection on a single chip format. In FIG. 4B, the biosensor for biological detection uses bacteriophage specificity to identify the target microorganism. Phage are genetically modified to contain a quorum sensing signal architecture (e.g., luxI), and, upon target host infection, instigate synthesis of autoinducer molecules within the host cell. A bioluminescent bioreporter responds specifically to the autoinducer and signals host cell presence via bioluminescence emission. Since each caged bioreporter population responds to its own unique autoinducer, chip quadrants registering positive signals can be pinpointed and used to identify the pathogen(s) present in the sample. Additional applications include the fabrication of microscale fluidic manifolds within the BBIC substrate for microfluidic input and output of sample (McKnight et al., Anal Chem. 73:4045-4049, 2001; McKnight et al., Nanotechnology 14:551-556, 2003.

### Kit for Detecting Microbes in a Sample

The invention includes a kit for detecting a target bacterium in a sample. The kit includes: (a) a recombinant bacteriophage having a nucleotide sequence encoding LuxI and capable of infecting the target bacterium, wherein the LuxI induces the production of at least one autoinducer molecule in the target bacterium when the bacteriophage infects the target bacterium, (b) at least one bioreporter bacterium having a nucleic acid encoding LuxR, a nucleic acid encoding LuxA, a nucleic acid encoding LuxB, a nucleic acid encoding LuxC, a nucleic acid encoding LuxD, and a nucleic acid encoding LuxE, the nucleic acids being operably linked to a promoter, and (c) instructions for using the recombinant bacteriophage and the at least one bioreporter bacterium. The kit can further include packaging, a solid substrate (e.g., microchip), and an integrated circuit. In preferred embodiments, the kit is portable for use outside of the laboratory.

### EXAMPLES

### Example 1

### Bioreporter system for E. coli using Phage Lambda

The feasibility of luxI-incorporated phage reporters was shown by constructing and testing a biodiagnostic system for E. coli using temperate phage lambda. The P_{L} promoter from phage lambda (GenBank accession no. J02459) was fused in-frame to a single V. fischeri luxI gene (accession no. M19039) followed by a T₁T₂ transcriptional terminator (accession no. X81837). The P_{L}-luxI-T₁T₂ construct was then inserted into the lambda genome, packaged into phage heads (Stratagene LambdaZAP and Gigapack kits), and propagated as luxI-bearing lambda phage (λ_{luxI}).

A bioluminescent bioreporter specific for OHHL was also constructed. This bioreporter, designated E. coli OHHLux, contains a chromosomal insert of the luxR regulatory gene and the complete luxCDABE gene cassette. It is capable of sensing OHHL down to 10 nM, and was used in the following experiment. λ_{luxI} reporter phage were combined at a multiplicity of infection (MOI) of 1000 with E. coli host cells at concentrations ranging from 0 to 1×10⁸ CFU/ml in 96-well microtiter plates containing minimal media. Each well was also inoculated with the OHHLux bioreporter at a concentration of approximately 1×10⁶ CFU/ml. Plates were incubated at room temperature in a Microbeta Victor2 Multilabel counter (Perkin-Elmer) with photon counts measured every 20 min. A dose-response profile was generated of the OHHLux bioreporter to OHHL synthesized by λ_{luxI} reporter phage infection of target E. coli host cells at concentrations ranging from 1 to 1×10⁸ cfu/ml. Significant bioluminescent responses were detected from 1 to 1×10⁴ E. coli cells within 8-11 hours post-inoculation. At cell densities >10⁴, detection could be achieved within 2-7 hours.

Detection of bacterial pathogens is based on the ability of OHHL molecules to induce bioluminescence in such a manner that it can be correlated back to the number of targets present in the sample. This approach uses the same principles as quantitative PCR with the exception that initial OHHL concentrations as opposed to nucleic acid concentrations allow for differential detection of the exponential increase in signal. Measuring the initiation of the geometric increase in bioluminescence allows for the quantification of target. The more phage infection events that occur, the higher the concentration of OHHL, thereby decreasing the time for autoinduction to occur. Measuring the time decrease between the control and the samples allows for the enumeration of the number of bacterial targets present.

### Example 2

Linking Bacteriphage infection to quorum sensing signaling and bioluminescent bioreporter monitoring for direct detection of bacterial agents.

### Materials and Methods

Bacterial strains and bacteriophages. The phage bioluminescent system includes three components; the luxI-incorporated reporter phage (λ_{luxI}), the AHL-specific bioluminescent bioreporter (E. coli OHHLux), and the target bacterium. The λ_{luxI} reporter phage was constructed within temperate phage lambda, lambda-resistant E. coli XLOLR (Stratagene, La Jolla, Calif.) was used for construction of the OHHL-specific bioluminescent bioreporter E. coli OHHLux, and the E. coli K12 variant XL1-Blue (Stratagene) was used as the model host strain for phage infection. lux genes were derived from V fischeri or Photorhabdus luminescens (Gupta et al., FEMS Yeast Res 4, 305-313, 2003). E. coli strains were typically grown in Luria-Bertani media (LB; 10 g tryptone, 5 g yeast extract, 10 g NaCL per 1 H₂O, pH 7.0). NZY top agar (5 g NaCL, 2 g MgSO₄.7H₂O, 5 g yeast extract, 10 g NZ amine, 7 g agarose per 1 H₂O, pH 7.0) was used to propagate and titer bacteriophage.

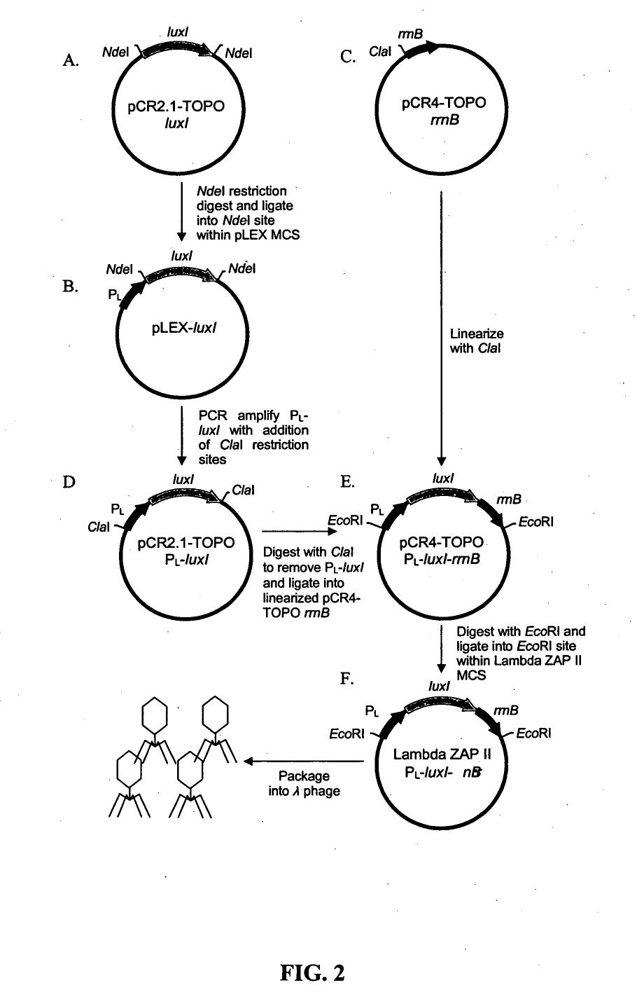

Genetic construction of the λ_{luxI} reporter bacteriophage. The fundamental construction of the luxI reporter phage involved a fusion of the V. fischeri luxI gene (GenBank accession no.Y00509) upstream of the left arm promoter (P_{L}) of phage lambda in a pLEX vector (Invitrogen, Carlsbad, Calif.) (FIG. 2). Upstream to this fusion was ligated an rrnB T₁T₂ transcriptional terminator from the pKK223-3 cloning vector (Accession #M77749). Each individual gene and step-wise fusions were initially constructed in pCR2.1- or pCR4-TOPO TA cloning vectors (Invitrogen) and then the entire fusion was ligated into the Lambda ZAP II cloning vector (Stratagene) and packaged into phage lambda using Gigapack III Gold packaging extract (Stratagene). DNA isolations were performed with Wizard Minipreps, Midipreps, or Lambda Preps (Promega, Madison, Wis.) and purified when necessary with the Geneclean Spin Kit (Q-Biogene, Carlsbad, Calif.). PCR reactions were carried out in an MJ Research DNA Engine tetrad (Waltham, Mass.) using Ready-To-Go PCR beads (Amersham Piscataway, N.J.). DNA was sequenced at all steps with the ABI Big Dye Terminator Cycle Sequencing reaction kit on an ABI 3100 DNA Sequencer (Perkin-Elmer, Foster City, Calif.). The luxI gene was PCR-amplified from V. fischeri using the primer pairs 5′-CATATGACTATAATGATAAAAAAATCGG (SEQ ID NO:1)-3′ and 5′-CATATGTTAATTTAAGACTGC (SEQ ID NO:2)-3′ to introduce the restriction site NdeI at both termini (underlined) and cloned into a pCR2.1-TOPO vector. (FIG. 2A). The luxI gene was then removed from the TOPO vector by NdeI restriction digestion and ligated into the NdeI multicloning site (MCS) of the pLEX vector, thereby placing luxI in frame with the P_{L} promoter (FIG. 2B). Directionality was confirmed by restriction digestion and sequencing.

The rrnB transcriptional terminator was PCR-amplified from pKK223-3 using the primer pairs 5′-ATCGATAAGAGTTTGTAGAAACGC (SEQ ID NO:3)-3′ and 5′CTGTTTTGGCGGATG (SEQ ID NO:4)-3′ to introduce the restriction site ClaI at the 5′ end (underlined) and cloned into a pCR4-TOPO vector (FIG. 2C).

The P_{L}-luxI fusion was PCR-amplified out of pLEX with the primer pairs 5′-ATCGATGTCGACTCTAGAGGATCC (SEQ ID NO:5)-3′ and 5′-ATCGATATTCGAGCTCGGTACCATA (SEQ ID NO:6)-3′ containing the restriction sites ClaI (underlined) and cloned into a pCR2.1-TOPO vector (FIG. 2D). This vector was then digested with ClaI and ligated into the ClaI site of the rrnB TOPO vector described above to create a P_{L}-luxI-rrnB fusion within a pCR4-TOPO vector (FIG. 2E). Directionality was again confirmed by restriction digestion and sequencing. Unique EcoRI sites within the multicloning site of the pCR4-TOPO vector now flanked the P_{L}-luxI-rrnB fusion, allowing for its removal via EcoRI digestion with subsequent ligation into the unique EcoRI site of the Lambda ZAP II vector (FIG. 2F). Resulting recombinant lambda phage DNA was then packaged into phage heads using Gigapack III packaging extract per the manufacturer's instructions (Stratagene). Resulting plaques were hybridized with an alkaline phosphatase-labeled luxI probe to chemifluorescently identify luxI-incorporated phage using an Alkphos Direct Labeling and Detection kit (Amersham). Positive plaques were then isolated and propagated on top agar plates as described in the Lambda ZAP II instructions to concentrations of approximately 1×10¹⁰ PFU ml⁻¹ and stored at 4° C.

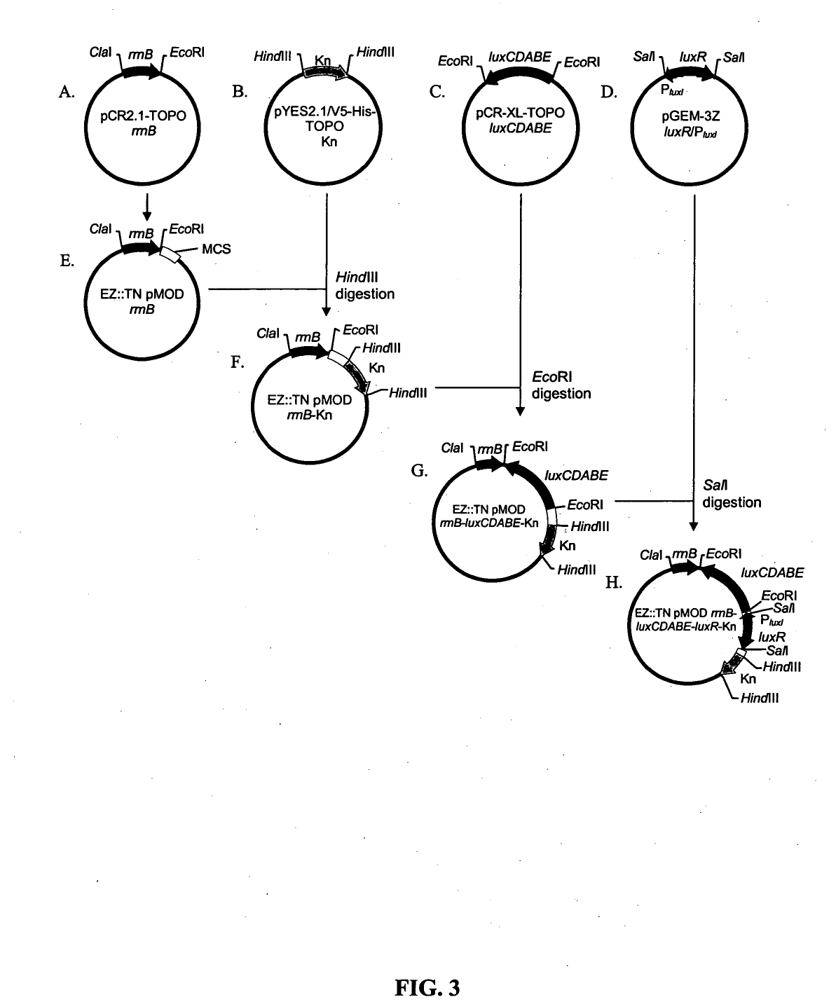

Genetic construction of E. coli OHHLux. The OHHL-specific bioluminescent bioreporter E. coli OHHLux was constructed by fusing the luxCDABE genes from P. luminescens with the luxR gene and luxI promoter region (P_{luxI}) from V. fischeri into an EZ::TN pMOD cloning vector (Epicentre Technologies, Madison, Wis.) in conjunction with an rrnB T₁T₂ transcriptional terminator and a kanamycin resistance gene (FIG. 3). The construct is similar to that of plasmid pSB401 created by Winson et al. (FEMS Microbiol Lett 163, 185-192, 1998), except the present construct design incorporates the cloning region on a hyperactive transposon theoretically capable of insertion into virtually any bacterial chromosome. The rrnB transcriptional terminator was PCR-amplified from pKK223-3 with the primer pairs 5′-ATCGATAAGAGTTTGTAGAAACGC (SEQ ID NO:7)-3′ and 5′-GAATTCCTGTTTTGGCGGATG (SEQ ID NO:8)-3′ containing the restriction sites ClaI and EcoRI (underlined) at the 5′ and 3′ ends, respectively, and cloned into a pCR2.1-TOPO vector (FIG. 3A). The kanamycin gene was PCR-amplified from a pCR2.1-TOPO vector with the primer pairs 5′-AAGCTTTCAGGGCGCAAGGGC (SEQ ID NO:9)-3′ and 5′-AAGCTTACTCTTCCTTTTTCAATTCAGAAGAAC (SEQ ID NO:10)-3′ containing terminal HindIII restriction sites (underlined) and cloned into a pYES2.1/V5-His-TOPO vector (Invitrogen) (FIG. 3B). The luxCDABE gene cassette was PCR-amplified from P. luminescens using the primer pairs 5′-ATTAAATGGATGGCAAATAT- (SEQ ID NO:11) 3′ and 5′-AGGATATCAACTATCAAAC (SEQ ID NO:12)-3′ and cloned into a pCR-XL-TOPO vector (Invitrogen) (FIG. 3C). The luxR gene and its neighboring P_{luxI} region were PCR-amplified from V. fischeri with the primer pairs 5′-GTCGACCCTATAGGTATAAAGCTTTACTTACG (SEQ ID NO:13)-3′ and 5′-GTCGACTACCAACCTCCCTTGCG (SEQ ID NO:14)-3′ containing SalI restriction sites at both termini (underlined) and cloned into a pGEM-3Z vector (Promega) digested with SalI (FIG. 3D). The rrnB TOPO clone was digested with ClaI and EcoRI and ligated into compatible ClaI and EcoRI sites within the MCS of the EZ::TN pMOD vector to create EZ::TN pMOD-rrnB (FIG. 3E). The kanamycin TOPO clone was digested with HindIII and ligated into the compatible HindIII site within the MCS of EZ::TN pMOD-rrnB to create EZ::TN pMOD-rrnB-Kn (FIG. 3F). The luxCDABE TOPO clone was digested with EcoRI (EcoRI sites are within the pCR-XL-TOPO MCS) and ligated into the compatible EcoRI site at the 3′ end of rrnB in the EZ::TN pMOD-rrnB-Kn vector to create EZ::TN pMOD-rrnB-luxCDABE-Kn (FIG. 3G). Directionality of the luxCDABE insert was confirmed by restriction digestion and sequencing. The luxR/P_{luxI} pGEM clone was digested with SalI and ligated into the SalI site of the MCS in EZ::TN pMOD-rrnB-luxCDABE-Kn to create EZ::TN pMOD-rrnB luxCDABE-luxR-Kn (FIG. 3H). Directionality of the luxR/P_{luxI} insert was confirmed by restriction digestion and sequencing. The EZ::TN pMOD rrnB-luxCDABE-luxR-Kn vector resides within E. coli XLOLR as a plasmid.

Dose response kinetics of the E. coli OHHLux bioreporter to OHHL. Synthetic OHHL (Sigma-Aldrich, St. Louis, Mo.; catalog no. K-3007) was diluted in 5 ml aliquots of M9 minimal media to desired concentrations and 100 μl of each dilution aliquot was added to triplicate wells in black 96-well microtiter plates (Dynex Technologies, Chantilly, Va.). E. coli OHHLux was grown in LB at 37° C. to an OD₆₀₀ of 0.6 (˜1×10⁸ CFU ml⁻¹) and 50 μl was added to each microtiter plate well containing the diluted OHHL. As well, E. coli OHHLux was added to wells void of OHHL to determine noninduced background levels of bioluminescence. Plates were sealed with transparent adhesive film (TopSeal-A, Perkin-Elmer, Boston, Mass.) and placed in a Perkin-Elmer Victor2 Multilabel counter at 30° C. with shaking (‘normal’ speed was selected with a 0.5 mm orbital diameter) with light collection programmed for 1 s well⁻¹ at 20 min intervals. In this and all experiments described below, resulting bioluminescent measurements were given the arbitrary light unit of counts s⁻¹ (CPS). A bioluminescent response was considered significant if it achieved an intensity 2 standard deviations (2a) above the negative control sample.

Specificity of the E. coli OHHLux bioreporter towards OHHL. Gram negative and Gram positive bacteria participate in quorum sensing communication networks via the production of many different types of AHL autoinducers or oligopeptides, respectively (Miller et al., Annu Rev Microbiol 55, 165-199, 2001). To demonstrate that the E. coli OHHLux bioreporter responded to OHHL and not to other autoinducers, the bacteria listed in TABLE 2 were grown in media and at temperatures specified by the American Type Culture Collection (ATCC) to an OD₆₀₀ of 0.6 and individually combined 1:1 in 96-well microtiter plates with E. coli OHHLux grown to an OD₆₀₀ of 0.6 in LB at 37° C. Wells containing only E. coli OHHLux served as negative controls for monitoring background levels of bioluminescence. Positive control wells contained E. coli OHHLux and 10 mmol 1⁻¹ synthetic OHHL (final volume). Plates were incubated in the Victor2 Multilabel counter at 30° C. with shaking with bioluminescence monitored for 1 s well⁻¹ at approximate 20 min intervals.

TABLE 2

| | Autoinducer, oligopeptide, | |
|---|---|---|
| Organism | or autoinducer lactonase | Reference |
| Agrobacterium | OOHL | Fuqua and Winans, J Bacteriol. |
| tumefaciens | | 176: 2796-3806, |
| ATCC 33970 | | 1994 |
| Arthrobacter | AHL lactonase | Park et al., Microbiol., |
| globiformis | | 149: 1541-1550, 2003 |
| KACC 10580 | | |
| Bacillus mycoides | AHL lactonase | Dong et al. Appl Environ |
| ATCC 37015 | | Microbiol. 68: 1754-1759, |
| | | 2002 |
| Burkholderia cepacia | OHL | Lewenza et al. J Bacteriol. |
| ATCC 25416 | | 181: 748-756, 1999 |
| Erwinia carotovora | OHHL | Pirhonen et al., EMBO J. |
| ATCC 15713 | | 12: 2467-2476, 1993 |
| Pseudomonas | OdDHL, BHL, AHL | Huang et al., Appl |
| aeruginosa | lactonase | Environ Microbiol., |
| ATCC BAA-47 | | 69: 5941-5949, 2003 |
| Rhodobacter | 7,8-cis-N-(tetradecanoyl)- | Puskas et al., J Bacteriol. |
| sphaeroides | homoserine lactone | 179: 7530-7537, 1997 |
| ATCC 55304 | | |
| Serratia liquefaciens | HHL, BHL | Eberl et al., Mol |
| ATCC 11367 | | Microbiol. 20: 127-136, |
| | | 1996 |
| Staphylococcus aureus | AIP (autoinducing peptide) | Mayville et al., Proc Natl |
| ATCC 35556 | | Acad Sci USA, 96: 1218-1223, |
| | | 1999 |
| Yersinia enterocolitica | HHL, OHHL | Throup et al., Mol |
| ATCC 23715 | | Microbiol. 17: 345-356, |
| | | 1995 |

OHHL, N-(3-oxohexanoyl)-homoserine lactone;
OdDHL, N-(3-oxododecanoyl)-homoserine lactone;
OHL, N-octanoyl-homoserine lactone;
OOHL, N-(3-oxooctanoyl)-homoserine lactone;
HHL, N-hexanoyl-homoserine lactone;
BHL, N-butanoyl-homoserine lactone.
For specifics on AHL lactonases and acylases, see Roche et al. (2004).

Phage reporter pure culture assay. To determine target cell detection limits, λ_{luxI} reporter phage and E. coli OHHLux were combined with a dilution series of E. coli XL1-Blue down to an estimated 1 CFU ml⁻¹. λ_{luxI} reporter phage were prepared on top agar overlays and stored at stock concentrations of 1×10¹⁰ PFU ml⁻¹. The OHHLux bioluminescent bioreporter was grown in LB at 37° C. to an OD₆₀₀ of 0.6 (˜1×10₈ CFU ml⁻¹) and used as is. The E. coli host XL1-Blue was grown at 30° C. in LB to an OD₆₀₀ of 0.7 (˜1×10⁹ CFU ml⁻¹) then diluted 1:10 down to approximately 1 CFU ml⁻¹ in 50 ml conical centrifuge tubes containing 9 ml LB. One hundred microliter aliquots of each XL1-Blue dilution were then distributed columnwise (100 μl well-1, 8 wells columns⁻¹) throughout a black 96-well microtiter plate. A control column received 100 μl of LB well⁻¹. To all wells was then added 100 μl of λ_{luxI} reporter phage stock (˜1×10⁹ PFU ml⁻¹ final concentration) and 50 μl of OHHLux bioreporter (˜5×10⁶ CFU ml⁻¹ final concentration). This equates to an approximate upper MOI of 10, and establishes a high infection rate of XL1-Blue cells. Plates were monitored for bioluminescence overnight in the Victor2 Multilabel counter (30° C., shaking, 1 s well⁻¹, 20 min intervals).

Each XL1-Blue dilution tube was additionally incubated in a standard laboratory incubator with shaking (200 rev min⁻¹) at 37° C. to promote better growth than that achievable in the microtiter plate. After 5 h, 100 μl aliquots were removed from the preincubated dilution tubes as well as from a preincubated LB control tube and transferred to a microtiter plate as described above, with λ_{luxI} reporter phage and OHHLux bioreporters added to each well also as described above. The microtiter plate was similarly monitored for bioluminescence. All dilutions of XL1-Blue, both at the beginning and after the 5 h incubation period, were plated in triplicate on LB agar containing tetracycline at 14 mg 1⁻¹ (LBTc) to determine viable cell counts. Each microtiter plate assay was also run with a duplicate control series of dilutions wherein E. coli XL1-Blue was replaced with the lambda resistant strain E. coli SOLR (Stratagene).

Lettuce leaf wash assays. E. coli XL1-Blue was grown at 30° C. in 200 ml LB to an OD₆₀₀ of 0.6 (˜1×10⁸ CFU ml⁻¹), centrifuged at 1000×g for 10 min, and resuspended in 200 ml sterile water. A 1:10 dilution series was then prepared in 200 ml volumes of sterile water to form E. coli-contaminated water ranging from 108 to approximately 1 CFU ml⁻¹. A control tube not receiving an XL1-Blue inoculum was also prepared. Grocery store-purchased iceberg head lettuce was rinsed with 1 liter sterile water and spun dry in a kitchen salad spinner (Zyliss Corp., Foothill Ranch, Calif.). Ten grams of lettuce were placed in each dilution of E. coli-contaminated water for 5 min with shaking (200 rev min⁻¹), spun dry in the salad spinner, and individually transferred to 30 ml of sterile saline. After 2 min of shaking (200 rev min⁻¹), saline diluents were transferred to 50 ml conical centrifuge tubes and centrifuged for 10 min at 3000×g. Resulting pellets were resuspended in 3 ml LB and then assayed in microtiter plates either immediately or after a 16 h preincubation at 37° C. with shaking (200 rev min⁻¹). Preincubation was performed under tetracycline selection (14 mg l⁻¹ final concentration) to select for XL1-Blue cells. Aliquots of 100 μl were removed from each LB resuspension either immediately or after the 16 h preincubation and transferred columnwise (100 μl well⁻¹, 8 wells column⁻¹, 1 column (dilution tube)⁻¹) to a 96-well black microtiter plate. Preincubated samples were washed once with LB to remove residual tetracycline just prior to sample transfer to the microtiter plate. Each well then received 100 μl of λ_{luxI} reporter phage stock and 50 μl of OHHLux bioreporter prepared as described above for the pure culture assays. Microtiter plates were sealed with adhesive and monitored for bioluminescence in the Victor2 Multilabel counter (30° C., shaking, 1 s well⁻¹, 20 min intervals). Each LB resuspension was plated in triplicate on LBTc plates both immediately and after the 16 h incubation to determine viable XL1-Blue cell counts.

Analytical measurement of OHHL. OHHL concentrations were analytically determined using a ThermoFinnigan LCQ DecaXPplus liquid chromatograph-mass spectrometer (LCMS) fitted with a 10 cm×4.6 mm id C18 column (Advanced Chromatography Technologies, Chadds Ford, Pa.). Triplicate culture supernatant aliquots ranging from 1 to 50 ml were extracted twice with equal volumes of ethyl acetate, dried under nitrogen, and redissolved in 1 ml of methanol. A flow rate of 0.2 ml min⁻¹ was used, starting with 20% methanol going to 95% methanol in 27 min with a 7 min hold, returning to 20% methanol in 6 min, and equilibrating for 5 min.

### Results

OHHL dose response of the E. coli OHHLux bioreporter. The E. coli OHHLux bioreporter was exposed to varying concentrations of synthetic OHHL to determine detection limits. Significant bioluminescent signals (2 s above background) were produced in response to OHHL at concentrations ranging from 10 nmol l⁻¹ to 50 μmol l⁻¹. Saturation-type behavior was observed at OHHL concentrations exceeding 50 μmol l⁻¹. A response linearity was demonstrated at the lower OHHL concentrations ranging from 20 nmol l⁻¹ to 2 μmol l⁻¹ (R2=0.99).

Specificity of the E. coli OHHLux bioreporter towards OHHL. The E. coli OHHLux bioreporter was co-cultured with bacterial strains synthesizing other classes of quorum sensing autoinducers or oligopeptides (TABLE 2). Significant bioluminescence was initiated only in response to the OHHL-synthesizing strains Erwinia carotovora and Yersinia enterocolitica, which generated bioluminescence at 87% and 64%, respectively, that of control wells containing E. coli OHHLux exposed to 10 nmol l⁻¹ synthetic OHHL. The remaining strains produced bioluminescence at less than 1% of the E. coli OHHLux control.

Phage reporter assay in pure culture. To test assay detection limits and response times, a 1:10 dilution series of target E. coli XL1-Blue cells ranging from approximately 1×10⁸ to 1 CFU ml⁻¹ was added to λ_{luxI} reporter phage and E. coli OHHLux bioreporters in 96-well microtiter plates both with and without a supplementary 5 h preincubation. Without preincubation, the microtiter plate assay was capable of detecting target E. coli XL1-Blue cells at an initial cell concentration, as determined by plate counts, of 1.1×10⁸ CFU ml⁻¹ within 1.5 h, 2.0×10⁷ CFU ml⁻¹ within 2.2 h, 1.2×10⁶ CFU ml⁻¹ within 3.6 h, and 2.9×10⁵ CFU ml⁻¹ within 4.9 h. XL1-Blue cell concentrations below 1 CFU ml⁻¹ did not generate significant bioluminescence. However, preincubating the dilution tubes at 37° C. with shaking for 5 h prior to initiation of the assay permitted better growth of XL1-Blue cells than in the constrained microtiter plate wells, and allowed for detection down to 1 (±2.5) CFU ml⁻¹ within a total assay time of 10.3 h. Duplicate control microtiter plates were also prepared substituting E. coli XL1-Blue with the lambda-resistant strain E. coli SOLR. No significant bioluminescence was observed in these plates.

Lettuce leaf rinse assays. Rinsings from iceberg lettuce artificially contaminated with a 1:10 dilution series of E. coli XL1-Blue cells were exposed to the phage reporter assay. At the highest average concentration of XL1-Blue cells (1.4×10⁸ CFU ml⁻¹), significant bioluminescence occurred within 2.6 h (TABLE 3). Successive 10-fold dilutions, yielding average cell concentrations of 1.5×10⁷, 1.3×10⁶, and 1.7×10⁵ CFU ml⁻¹, generated significant bioluminescence within 3.3, 10.3, and 12.1 h, respectively. Cell concentrations below 10⁵ CFU ml⁻¹ did not produce significant bioluminescence. Therefore, these dilutions were preincubated under tetracycline selection for 16 h to increase target cell concentrations, and then tested in the phage reporter assay. After the 16 h preincubation, the control tube, void of an XL1-Blue inoculum, indicated a background concentration of nontarget tetracycline resistant cells of 3.6×10⁸ CFU ml⁻¹. Estimated concentrations of tetracycline-resistant XL1-Blue cells within this background population were enumerated in each dilution tube based on similar colony morphology and are listed in TABLE 3. With selective overnight incubation, the original 10⁴ and 10³ inoculums of XL1-Blue cells could be detected within a total assay time, including the 16 h preincubation, of 19.1 h. The original 10² inoculum was detectable within 22.4 h. No significant bioluminescence was observed from XL1-Blue dilutions lower than 1×10² CFU ml⁻¹.

TABLE 3
Detection of E. coli XL1-Blue in leaf lettuce wash assays

| | Estimated E. coli XL1- | | |
|---|---|---|---|
| Initial E. coli XL1-Blue | Blue concentration after | Time until | Peak |
| inoculum (CFU | 16 h pre-incubation | bioluminescence | bioluminescence |
| ml⁻¹)* | (CFU ml−1) | induction (h) | (CPS) |
| 1.4 × 10⁸ | NA† | 2.6 | 817,000 |
| 1.5 × 10⁷ | NA | 3.3 | 31,500 |
| 1.3 × 10⁶ | NA | 10.3 | 8,600 |
| 1.7 × 10⁵ | NA | 12.1 | 6,800 |
| 1.7 × 10⁴ | 3.8 × 10⁸ | 19.1‡ | 81,800 |
| 1.5 × 10³ | 2.0 × 10⁸ | 19.1‡ | 59,750 |
| 130 (±21) | 1.1 × 10⁷ | 22.4‡ | 17,300 |
| 20 (±4) | 9.6 × 10⁴ | ND§ | ND |
| 2 (±3) | 6.6 × 10⁴ | ND | ND |

*Iceberg lettuce (10 g) was washed with water artificially contaminated at levels indicated
†NA, not assayed
‡Includes a 16 h preincubation prior to initiation of the assay
§ND, not detected

### Example 3

### Increasing sensitivity to bacterial pathogens

To increase sensitivity, autoinducer synthesis can be increased, and this can be done by integrating multiple luxI genes within the phage. High-level expression of luxI and corresponding high-level synthesis of OHHL autoinducer instigates a faster response from the E. coli OHHLux bioreporters during low-level target exposure. The same system as described in FIG. 2 is used. The luxI gene is PCR-amplified from V. fischeri (GenBank accession no. AF074719) using the primer pair 5′-CATATGACCGGTACTATAATGATAAAAAAATCGG (SEQ ID NO:15)-3′ and 5′-ACGCGTTCCGGATTAATTTAAGACTGC (SEQ ID NO:16)-3′ containing unique tandem restriction sites at each end (underlined) and cloned into a pCR2.1 TOPO vector. The terminal tandem restriction sites allow for directional insertion of additional luxI genes. For example, the PCR products above generate the sequence NdeI-AgeI-luxI-MluI-BspEI. A second luxI gene can be PCR-amplified with the sequence NdeI-luxI-AgeI and ligated in front of the first luxI gene to create NdeI-luxI-AgeI-luxI-MluI-BspEI. Another luxI gene with the added restriction sites of MluI-luxI-XmaI-BspEI can be inserted at the end via ligation between the MluI and BspEI sites to create NdeI-luxI-AgeI-luxI-MluI-luxI-XmaI-BspEI. The terminal XmaI and BspEI sites provide the next insertion site to create, for example, NdeI-luxI-AgeI-luxI-MluI-luxI-XmaI-luxI-SmaI-BspEI. By continuously adding tandem restriction sites to the 3′ end of the sequence, one can continuously add luxI genes. The final luxI gene contains an NdeI-BspEI site to position an NdeI site at the 3′ end. Along with the previously inserted NdeI site at the 5′ end, these flanking NdeI sites can then be used as described in FIG. 2 to create a P_{L}-multiluxI-rrnB construct that can be packaged into phage lambda. To determine the optimal number of luxI copies, constructs containing 2, 3, 4, 5 or more, luxI copies are created and individually tested and compared to determine optimum expression within the assay format.

### Example 4

### luxI-Integrated B40-9 Reporter Phage for the Detection of B. fragilis

To construct a reporter phage for the detection of B. fragilis, the luxI gene is first isolated and placed within a cloning vector for subsequent insertion into phage B40-8. Using standard PCR techniques, primers with unique restriction site overhangs are designed to amplify the luxI gene from V. fischeri. The resulting fragment is cloned into the broad host range vector pKBF367-1, which can be expressed in E. coli as well as B. fragilis. Transformants are subjected to restriction analysis for verification of insert size and orientation. Strains containing the correct construct are screened for the production of the diffusible OHHL signal by testing the supernatant for induction activity using the OHHLux bioluminescent bioreporter strain described above.

The assay is conducted by growing the B. fragilis cultures containing the correct construct to an optical density of 1.0 at 546 nm followed by centrifugation. The supernatant is tested by adding aliquots to the OHHLux strain and monitoring bioluminescence output. Clones producing OHHL are sequenced for verification. The functional luxI gene is then inserted into phage B40-8 through homologous recombination. The basic strategy follows that of the A511::luXAB phage described by Loessner et al. (1996) in “Construction of luciferase reporter bacteriophage A511::luxAB for rapid and sensitive detection of viable Listeria cells”, of Appl Environ Microbiol, 62, 1133-1140 and the lambda::luxAB fusions created by Duzhii and Zavilgelskii (1994). The luxI construct developed as described above is amplified with a set of primers containing flanking DNA sequences 50 bp downstream of the 3′ end of the major tail gene of phage B40-8 (GenBank accession no. AF074719) as well as a set of unique tandem restriction sites. This construct is inserted into a pKBF367-1 vector with PCR-modified T₁T₂ termination signals containing 5′ flanking sequences homologous to the direct 3′ ends of the tail gene. The product is amplified and subsequently inserted into the phage by recombination as previously described (Duzhii and Zavilgelskii, Mol Gen Mikrobiol Virusol 3:36-38, 1994; Loessner et al., Appln Environ Microbiol 62, 1133-1140, 1996). This yields recombinant phage containing a single luxI gene that is then tested for functionality. The unique tandem restriction sites at the 3′ end allow for successive insertion of more luxI genes in the same manner as described above for the λ_{luxI} reporter phage. Therefore, once the single insert luxI clone is proven functional, stepwise insertion of additional luxI genes occurs within the pKBF367-1 vector followed by recombination into the phage genome.

### Example 5

### High-Throughput Testing in Buffered Media

To greatly simplify and accelerate the process of testing multiple luxI-integrated phage constructs, a Biomek high-throughput liquid handling system (Beckman) integrated with a Victor2 bioluminescent reader (Perkin-Elmer) and liquid chromatograph/mass spectrometer (LCMS; ThermoFinnigan) can be used. Recombinant phage are screened and enriched in 96-well microtiter plates using a modification of the protocol of Loessner et al. (Appl Environ Microbiol 62, 1133-1140, 1996). Each phage construct is first incubated with its appropriate host (E. coli K12 (ATCC 29425) or B. fragilis HSP40 (ATCC 51477)) and OHHLux bioreporter cells in buffered media. All constructs that fail to produce adequate bioluminescent responses are eliminated. Although B. fragilis HSP40 is anaerobic, it does not require handling under strict anaerobic conditions. It is sufficient to fill wells and then cover with a film of plate sealer (Araujo et al., J Oral Maxillofac Surg. 59:1034-1039, 2001,). This is performed at various phage, host, and reporter dilution ranges to ensure that each component of the assay is optimally supplied. Selected constructs are then tested within a more defined dilution range to determine lower detection limits. Standard plate methods are used to determine target cell, bioreporter, and phage counts. Standard LCMS techniques are used for analytical measurement of OHHL production (Camara et al., Methods Microbiol 27, 319-330, 1998). Background bioluminescence due to basal level expression of the lux gene is determined in microtiter plates containing only the OHHLux bioreporter. Plotting background-corrected bioluminescence versus time generates standard curves indicating detection limits and response times. A negative control consisting of samples void of phage is used to account for intrinsic OHHL production.

### Example 6

### Bacterial Testing

The E. coli and B. fragilis assays described above are applied to tap water and freshwater obtained from local streams within the 96-well format described above, using similar controls and similar analytic measurements. Results are validated against standard molecular and morphological detection methods for E. coli and B. fragilis to assess sensitivity and minimum detection limits. The regulatory acceptable limit for recreational water uses when measuring E. coli is 126 CFU/100 ml. Thus, the target sensitivity for the E. coli phage-based biosensor is at least 1 CFU/ml. The EPA-approved ColiBlue24TM Test assay (MEL/MF total coliform lab, HACH Company, Ames, I A) can be used on all field samples for the enumeration of E. coli. In this objective, parallel samples are tested using the E. coli ColiBlue24TM Test and the E. coli phage-based assay as described above. Sample testing for both assays progresses from 1) serial dilutions of E. coli in tap water to 2) serial dilutions of human feces in tap water. Data is compared to determine the sensitivity of each assay.

Because the E. coli phage-based assay's original host is a laboratory-based E. coli, the infectivity range of the phage biosensor is tested against E. coli strains recently isolated from the environment. These strains are isolated from local water samples, which appear as blue colonies on the ColiBlue24TM filters. Non-E. coli strains, which should not be infected by the phage biosensor, appear as red colonies on the ColiBlue24TM filters and are also cultured. The E. coli strains are verified by standard phenotypic tests.

### Example 7

### System for Detecting Bacteroides in a Sample

An example of a bacterium, in addition to E. coli, that can be detected using the system of the invention is Bacteroides fragilis. Bacteroides is useful as a fecal bacterial indicator because it is the dominant bacterium in feces (up to 30% of the population) and may comprise approximately 10% of the fecal mass. In addition, Bacteroides species are animal host-specific (Bernhard and Field, Appl Environ Microbiol. 66:4571-4574, 2000), thus making them attractive targets for differentiating human and non-human sources of fecal contamination. B. fragilis is predominantly isolated from human feces and not other animal feces, so its presence in the environment may signal human fecal contamination as opposed to fecal contamination from other animals. To construct a phage-based assay for detecting Bacteroides, one or more (e.g., multiple) luxI gene constructs can be incorporated into the species-specific B. fragilis bacteriophage B40-8 and E. coli bacteriophage lambda for infection-inducible expression of quorum sensing signaling molecules (autoinducers). The efficacy of the assay can be tested with an autoinducer-sensing bioluminescent (lux) bioreporter. The phage infection and bioreporter effectiveness can be evaluated in detection studies within tap water and surface freshwater artificially contaminated with B. fragilis, E. coli, and/or human feces. By using E. coli and Bacteroides in tandem, these two sensors allow a direct comparison of data collected using the phage-based sensors with regulatory accepted plate culturing methods and allows the discrimination between fecal contamination attributable to human and non-human animal sources.

### Other Embodiments

While the above description contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A method for detecting a target bacterium in a sample, the method comprising the steps of:
(a) contacting the sample with a recombinant bacteriophage that is capable of infecting the target bacterium, the recombinant bacteriophage comprising a nucleotide sequence encoding a molecule capable of upregulating synthesis of at least one autoinducer molecule in the target bacterium;
(b) contacting at least a portion of the sample that has been contacted with the recombinant bacteriophage with at least one bioreporter bacterium comprising (i) a receptor capable of specifically binding the at least one autoinducer molecule and (ii) a nucleic acid encoding a reporter molecule;
(c) placing the at least a portion of the sample that has been contacted with the at least one bioreporter bacterium under conditions that promote (i) the expression of and diffusion of the at least one autoinducer molecule from the target bacterium and (ii) the uptake of the at least one autoinducer molecule by the at least one bioreporter bacterium; and
(d) detecting expression of the reporter molecule in the at least one bioreporter bacterium, wherein expression of the reporter molecule indicates that the target bacterium was present in the sample.

2. The method of claim 1, wherein the reporter molecule comprises LuxA and LuxB.

3. The method of claim 1, wherein binding of the at least one autoinducer molecule to the receptor upregulates expression of the nucleic acid encoding a reporter molecule.

4. The method of claim 1, wherein the molecule capable of upregulating synthesis of at least one autoinducer molecule in the target bacterium is LuxI, and the receptor that specifically binds the at least one autoinducer is LuxR.

5. The method of claim 1, wherein the at least one bioreporter bacterium further comprises a nucleic acid encoding LuxC operably linked to at least one promoter, a nucleic acid encoding LuxD operably linked to at least one promoter, and a nucleic acid encoding LuxE operably linked to at least one promoter.

6. The method of claim 1, wherein the amount of reporter molecule expression is proportional to the amount of target bacteria in the sample.

7. The method of claim 1, wherein the target bacterium is a food pathogen.

8. The method of claim 7, wherein the target bacterium is Escherichia coli.

9. The method of claim 1, wherein the sample is selected from the group consisting of: water, food, and water that has contacted food.

10. The method of claim 1, wherein the recombinant bacteriophage is phage lambda.

11. The method of claim 1, wherein the at least one bioreporter bacterium is Escherichia coli.

12. The method of claim 1, wherein the autoinducer molecule is an acyl-homoserine lactone.

13. The method of claim 12, wherein the acyl-homoserine lactone is N-3-(oxohexanoyl)-L-homoserine lactone.

14. The method of claim 1, wherein the recombinant bacteriophage further comprises at least three copies of the nucleotide sequence encoding a molecule capable of upregulating synthesis of at least one autoinducer molecule.

15. The method of claim 1, wherein the recombinant bacteriophage further comprises at least seven copies of the nucleotide sequence encoding a molecule capable of upregulating synthesis of at least one autoinducer molecule.

16. A kit for detecting a target bacterium in a sample, the kit comprising:
(a) a recombinant bacteriophage that is capable of infecting the target bacterium, the recombinant bacteriophage comprising a nucleotide sequence encoding a molecule capable of upregulating synthesis of at least one autoinducer molecule in the target bacterium; and
(b) instructions for using the recombinant bacteriophage in conjunction with at least one bioreporter bacterium comprising (i) a receptor capable of specifically binding the at least one autoinducer molecule and (ii) a nucleic acid encoding a reporter molecule.

17. The kit of claim 16, wherein the kit further comprises (c) at least one bioreporter bacterium comprising (i) a receptor capable of specifically binding the at least one autoinducer molecule and (ii) a nucleic acid encoding a reporter molecule,
wherein expression of the reporter molecule indicates the presence of the target bacterium in the sample.

18. The kit of claim 17, wherein the reporter molecule comprises LuxA and LuxB.

19. The kit of claim 16, wherein the molecule capable of upregulating synthesis of at least one autoinducer molecule in the target bacterium is LuxI, and the receptor that specifically binds the at least one autoinducer molecule is LuxR.

20. The kit of claim 16, wherein the target bacterium is a food pathogen.

21. The kit of claim 20, wherein the target bacterium is Escherichia coli.

22. The kit of claim 16, wherein the recombinant bacteriophage is phage lambda.

23. The kit of claim 16, wherein the at least one bioreporter bacterium is Escherichia coli.

24. The kit of claim 16, wherein the at least one bioreporter bacterium is resistant to infection by the recombinant bacteriophage.

25. The kit of claim 16, wherein the autoinducer molecule is an acyl-homoserine lactone.

26. The kit of claim 25, wherein the acyl-homoserine lactone is N-3-(oxohexanoyl)-L-homoserine lactone.

27. The kit of claim 16, wherein the molecule that is capable of upregulating synthesis of at least one autoinducer molecule in the target bacterium upregulates synthesis of a plurality of autoinducer molecules in the target bacterium.

28. The kit of claim 17, wherein binding of the at least one autoinducer molecule to the receptor upregulates expression of the nucleic acid encoding the reporter molecule.

29. A kit for detecting a target bacterium in a sample, the kit comprising: a solid substrate having a plurality of bioreporter bacteria disposed thereon, each bioreporter bacterium comprising (i) a receptor capable of specifically binding the at least one autoinducer molecule and (ii) a nucleic acid encoding a reporter molecule, the bioreporter bacteria being in operable proximity to an integrated circuit for detecting and quantitating expression of the reporter molecule, and instructions for use of the kit with a recombinant bacteriophage that is capable of infecting the target bacterium, the recombinant bacteriophage comprising a nucleotide sequence encoding a molecule capable of upregulating synthesis of at least one autoinducer molecule in the target bacterium.

30. The kit of claim 29, wherein the molecule capable of upregulating synthesis of at least one autoinducer molecule in the target bacterium upregulates synthesis of a plurality of autoinducer molecules in the target bacterium.

31. The kit of claim 29, wherein the solid substrate is a microchip.

32. The kit of claim 29, wherein the kit is portable.

33. The kit of claim 29, wherein the amount of reporter molecule expression is proportional to the amount of target bacteria in the sample.

34. A kit for detecting a target bacterium in a sample, the kit comprising: a solid substrate having a plurality of bioreporter bacteria disposed thereon, each bioreporter bacterium comprising (i) a receptor capable of specifically binding the at least one autoinducer molecule and (ii) a nucleic acid encoding a reporter molecule, the bioreporter bacteria in operable proximity to at least one photodetector for detecting expression of the reporter molecule, the photodetector in operable engagement with at least one processor for storing information pertaining to the expression of the reporter molecule.


SEQUENCE LISTING (16 sequences)

SEQ ID NO: 1 - DNA, 28 bp, ARTIFICIAL
  OLIGONUCLEOTIDE
catatgactataatgataaaaaaatcgg

SEQ ID NO: 2 - DNA, 21 bp, ARTIFICIAL
  OLIGONUCLEOTIDE
catatgttaatttaagactgc

SEQ ID NO: 3 - DNA, 24 bp, ARTIFICIAL
  OLIGONUCLEOTIDE
atcgataagagtttgtagaaacgc

SEQ ID NO: 4 - DNA, 15 bp, ARTIFICIAL
  OLIGONUCLEOTIDE
ctgttttggcggatg

SEQ ID NO: 5 - DNA, 24 bp, ARTIFICIAL
  OLIGONUCLEOTIDE
atcgatgtcgactctagaggatcc

SEQ ID NO: 6 - DNA, 25 bp, ARTIFICIAL
  OLIGONUCLEOTIDE
atcgatattcgagctcggtaccata

SEQ ID NO: 7 - DNA, 24 bp, ARTIFICIAL
  OLIGONUCLEOTIDE
atcgataagagtttgtagaaacgc

SEQ ID NO: 8 - DNA, 21 bp, ARTIFICIAL
  OLIGONUCLEOTIDE
gaattcctgttttggcggatg

SEQ ID NO: 9 - DNA, 21 bp, ARTIFICIAL
  OLIGONUCLEOTIDE
aagctttcagggcgcaagggc

SEQ ID NO: 10 - DNA, 33 bp, ARTIFICIAL
  OLIGONUCLEOTIDE
aagcttactcttcctttttcaattcagaagaac

SEQ ID NO: 11 - DNA, 20 bp, ARTIFICIAL
  OLIGONUCLEOTIDE
attaaatggatggcaaatat

SEQ ID NO: 12 - DNA, 19 bp, ARTIFICIAL
  OLIGONUCLEOTIDE
aggatatcaactatcaaac

SEQ ID NO: 13 - DNA, 32 bp, ARTIFICIAL
  OLIGONUCLEOTIDE
gtcgaccctataggtataaagctttacttacg

SEQ ID NO: 14 - DNA, 23 bp, ARTIFICIAL
  OLIGONUCLEOTIDE
gtcgactaccaacctcccttgcg

SEQ ID NO: 15 - DNA, 34 bp, ARTIFICIAL
  OLIGONUCLEOTIDE
catatgaccggtactataatgataaaaaaatcgg

SEQ ID NO: 16 - DNA, 27 bp, ARTIFICIAL
  OLIGONUCLEOTIDE
acgcgttccggattaatttaagactgc